(19) United States
(12) Patent Application Publication
(10) Pub. No.: US 2009/0024140 A1
(43) Pub. Date: Jan. 22, 2009

(54) Surgical feedback system

(75) Inventors: Paul G. Allen, Seattle, WA (US); Edward Boyden, Cambridge, MA (US); W. Daniel Hillis, Encino, CA (US); Roderick A. Hyde, Redmond, WA (US); Muriel Y. Ishikawa, Livermore, CA (US); Edward K.Y. Jung, Bellevue, WA (US); Eric C. Leuthardt, St. Louis, MO (US); Nathan P. Myhrvold, Medina, WA (US); Dennis J. Rivet, St. Louis, MO (US); Michael A. Smith, Phoenix, AZ (US); Clarence T. Tegreene, Bellevue, WA (US); Thomas A. Weaver, San Mateo, CA (US); Charles Whitmer, North Bend, WA (US); Lowell L. Wood, JR., Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/880,432

(22) Filed: Jul. 20, 2007

(51) Int. Cl. A61B 19/00

(52) U.S. Cl. 606/130

(57) ABSTRACT

The invention pertains to a surgical system. In an embodiment, the system comprises one or more sensors operably responsive to physical boundary limitations of an operating field. Furthermore, the one or more sensors provide information regarding the physical boundary limitations of the operating field. In another embodiment, the system comprises a surgical instrument that is configured to respond to the information by either activation or inactivation. In another aspect the invention includes generating an anatomical image or anatomic positional reference data. Additionally, the method includes creating haptic feedback signals based at least partly on the anatomical image or anatomic positional reference data, and determining a position or orientation of a surgical instrument. Furthermore the method includes activating or inactivating the surgical instrument based at least partly on the haptic feedback signals.

### TECHNICAL FIELD

The present disclosure relates, in general, to devices, methods or systems for surgical treatment or management of disease, disorders, or conditions using feedback systems.

### SUMMARY

The present disclosure relates to a surgical system. In one aspect of the invention the surgical system comprises a feedback system having one or more sensors operably responsive to physical boundary limitations of an operating field. Furthermore, the one or more sensors provide information regarding the physical boundary limitations of the operating field. Additionally, a surgical instrument is provided that responds to the information by either activation or inactivation. In an embodiment, the activation or the inactivation can occur either within the physical boundary limitations of the operating field or outside the physical boundary limitations of the operating field. In a further embodiment, the activation or the inactivation includes autoactivation or autoinactivation of the surgical instrument. In another embodiment, the activation or the inactivation can occur through modification of one or more operative characteristics of the surgical instrument. In another embodiment, at least one or more sensors provide information regarding the physical boundary limitations of the operating field including boundary-sensing signals. In another embodiment, the boundary-sensing signals are delivered to the surgical instrument. Additionally, the boundary-sensing signals may be delivered to the surgical instrument via a direct connection that includes a hardwired system. Alternatively, the boundary-sensing signals can be delivered to the surgical instrument via a wireless system. The communication systems include any appropriate signal-carrying path or device such as for example, an optical fiber, a waveguide, a nanotube, a metal wire or a nonmetallic wire.

In another aspect of the present invention, a surgical instrument can be activated or inactivated while the surgical instrument is at least partly functioning within an operating field. Additionally or alternatively, at least one or more sensors can determine at least one orientation or position of the surgical instrument relative to the operating field. Furthermore, the at least one or more sensors can determine at least one orientation or position of the surgical instrument relative to a human or robotic user. In an additional embodiment, the surgical instrument may be optionally activated or inactivated via operable communication with a global positioning system. In a further embodiment, the surgical instrument may include at least one of the following devices: an endoscope, a dissector, a scalpel, a laser scalpel, a knife, a blade, a needle, a catheter, a scissors, a cutter, a grasper, a surgical tool, a driver, a drill, a saw, a clamper, a pulverizer/crusher, a grinder, a trocar device, a suturer or a stapler.

A further aspect includes a method of performing haptic surgery, which comprises at least one or more of the following steps: (1) generating an anatomical image or anatomic positional reference data; (2) creating haptic feedback signals based at least partly on the anatomical image or anatomic positional reference data; (3) determining a position or orientation of a surgical instrument; and (4) activating or inactivating the surgical instrument based at least partly on the haptic feedback signals. In an embodiment, the step of generating an anatomical image or anatomic positional reference data includes positioning one or more sensors in an operating field. Furthermore, the generating an anatomical image or anatomic positional reference data can include collecting one or more static images or anatomic positional reference data from an operating field. In an alternative embodiment, the generating an anatomical image or anatomic positional reference data includes collecting one or more dynamic images or anatomic positional reference data from an operating field. An embodiment provides that the step of determining a position or orientation of a surgical instrument includes positioning at least one or more sensors in an operating field by moving the at least one or more sensors from a first position proximate to a bodily tissue to a second position proximate to the bodily tissue.

In a further embodiment, a step of creating haptic feedback signals is partly based on near real time anatomical imaging. Alternatively or additionally, the step of creating haptic feedback signals may be partly based on a recorded anatomical imaging history. In a further embodiment, the step of creating haptic feedback signals includes converting one or more images or anatomic positional reference data from an operating field into one or more of haptic category objects. In another embodiment, the step of creating haptic feedback signals include converting one or more haptic category objects into one or more of haptic cues. The step of creating haptic feedback signals may optionally include binning the one or more haptic cues. Further embodiments may comprise of the haptic category objects being made available to a user in real time or nearly in real time. Additionally or alternatively, the step of creating haptic feedback signals includes making the haptic cues available to a user in real time or nearly in real time. An embodiment provides that the step of creating haptic feedback signals includes tactilely informing users of a distribution of forces being imposed on at least a portion of the surgical instrument. An alternative embodiment calls for the step of creating the haptic feedback signals as a function of one or more sensor signals. Furthermore, the step of creating haptic feedback signals include either scaling up or scaling down the one or more sensor signals in a linear or non-linear fashion. In another embodiment, the step of creating haptic feedback signals includes developing a database of reference haptic cues for a given operating field. Another embodiment provides for the step of creating haptic feedback signals that include developing a database of reference haptic cues from a texture map of an operating field. Another embodiment provides for the step of creating haptic feedback signals that include developing a database of reference haptic cues from a color map of an operating field. There is provided an embodiment in which, the step of creating haptic feedback signals includes implanting a plurality of fiducials within one or more images or anatomic positional reference data of the operating field proximate to one or more haptic objects.

Another embodiment of a method of performing haptic surgery comprises a step of activating or inactivating a surgical instrument. This step may optionally include performing a linear or affine transformation on at least one force measurement on an anatomical tissue. Furthermore, the step of activating or inactivating the surgical instrument may include applying instrument gain greater or less than unity during a contour mapping, a color mapping, a force measurement or a texture translating. In addition, the step of activating or inactivating the surgical instrument can include applying both tractor and non-tractor pressure stresses to portions of anatomical tissues. Alternative embodiments may include applying both tractor and non-tractor pressure stresses to portions of anatomical tissues. In an embodiment, the step of activating or inactivating the surgical instrument includes applying statically driven force points at different force levels to portions of anatomical tissue to obtain haptic feedback signals. Another embodiment provides for the step of activating or inactivating the surgical instrument to include applying binding and de-binding attachments to force points. Another embodiment may include the step of activating or inactivating the surgical instrument by the application of electrical or magnetic forces. In another embodiment the step of activating or inactivating the surgical instrument includes motions producing a realistic operating field manipulating environment. In accordance with an embodiment, the step of activating or inactivating the surgical instrument includes eliminating abrupt transactions between adjoining anatomical tissues by dynamically modifying a configuration of at least one body part in response to the at least one feedback signal. A further embodiment includes the step of activating or inactivating the surgical instrument that provides one or more haptic cues based in part on a haptic object. A further alternative embodiment provides that the step of activating or inactivating the surgical instrument includes modifying at least one portion of anatomical tissue in accordance with a therapeutic protocol. Another embodiment includes the step of activating or inactivating the surgical instrument while penetration of a haptic object by the surgical instrument. In another embodiment, the step of activating or inactivating the surgical instrument includes statically modifying at least one of a plurality of anatomical tissues in an operating field. Alternative embodiments can include dynamically modifying at least one of a plurality of anatomical tissues in an operating field. In one embodiment, the step of activating or inactivating the surgical instrument includes use of a patient monitoring information system or a hospital information system that may be coupled via a wired or a wireless means to the surgical instrument. Furthermore, the step of activating or inactivating the surgical instrument may also include user-initiated commands, which are in part based on a patient monitoring system or a hospital information system. In an alternative embodiment, the step of activating or inactivating the surgical instrument may include reorientation, reconfiguration, adjustment or repositioning of the surgical instrument. The reorientation or repositioning may occur through interlinking of a user interface with the surgical instrument or with an instrument positioning system or with any appropriate instrument tracking system. The positioning of the surgical instrument may be guided or facilitated by implanting fiducials in one or more locations within or outside an operating field. For example, fiducials could be placed through the use of or within a stereotactic surgical device, a sheet that may be used to cover the patient's body or parts thereof or other types of operating room landmarks. In an embodiment, the fiducials may function to facilitate activation or inactivation of the surgical instrument. Alternatively or additionally, the fiducials may be used to orient the surgical instrument in space and time in the operating field.

One embodiment provides that the step of activating or inactivating the surgical instrument includes activating or inactivating by a human user. Additionally or alternatively, the step of activating or inactivating the surgical instrument includes the step of activating or inactivating by a robotic user.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of a system level view of a surgical system;

FIG. 2 is an example of an operational flow for performing haptic surgery;

FIG. 3 is an illustration of an embodiment of a method of performing haptic surgery;

FIG. 4 is an example of an operational flow for performing haptic surgery;

FIG. 5 is an example of an operational flow for performing haptic surgery;

FIG. 6 is an example of an operational flow for performing haptic surgery; and

FIG. 7 is an example of an operational flow for performing haptic surgery.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

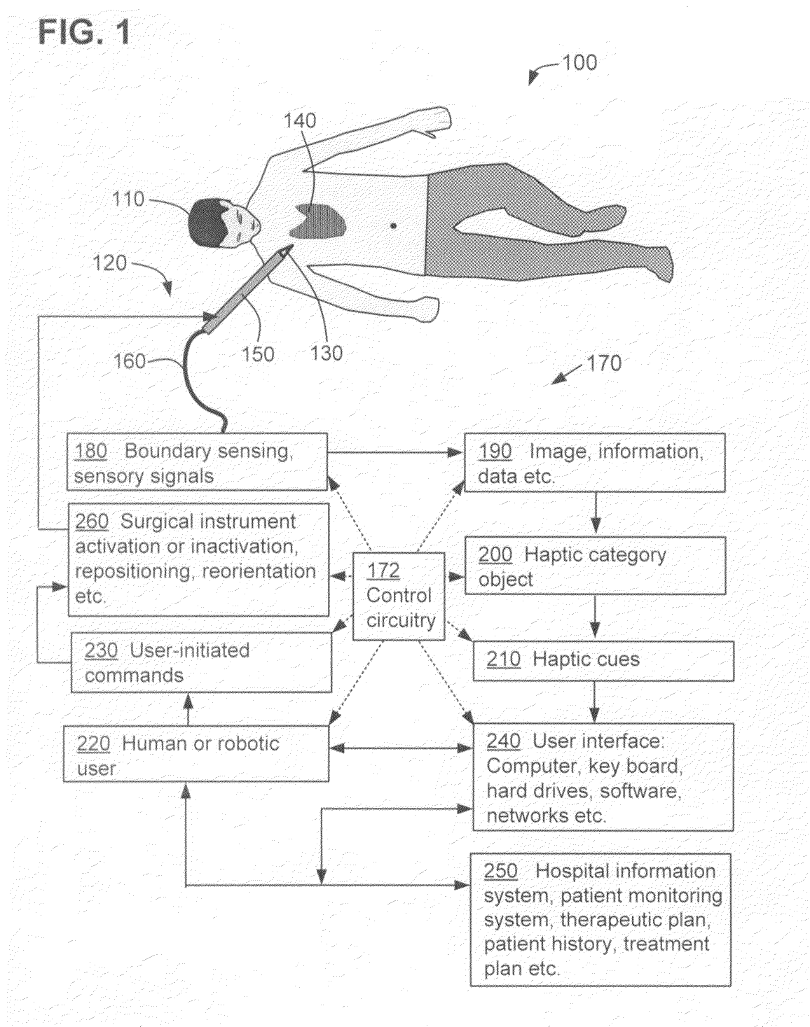

FIG. 1 is a schematic illustration of the surgical system 100, which, inter alia, comprises a patient 110 in need of a medical treatment or therapeutic management, and a feedback system 120. Those skilled in the art will appreciate that humans may be one of many categories of patients. Thus other patients may be envisaged, including, but are not limited to, an animal, a robotic simulator of a human or animal (e.g., computational entity), and/or substantially any combination thereof (e.g., a human or an animal patient may be assisted by one or more robotic agents). In addition, a human patient, although shown as a single person, may include other humans or non-human entities.

A feedback system 120 (see FIG. 1) may comprise of at least one or more sensors 130 operably responsive to physical boundary limitations of an operating field 140. In an embodiment, the at least one or more sensors may be operably coupled to a surgical instrument 150. As used herein, the surgical instrument may include, but is not limited to at least one or more of the following devices: an endoscope, a dissector, a scalpel, a laser scalpel, a knife, a blade, a needle, a catheter, a scissor, a cutter, a grasper, a surgical tool, a driver, a drill, a saw, a clamper, a pulverizer/crusher, a grinder, a trocar device, a suturer or a stapler, a sucker, a suction device, a cauterizing instrument, a retractor or a probe. In an alternative embodiment, at least one or more sensors 130 (see FIG. 1) may be component parts of any other device(s) used in an operating room. Additionally or alternatively, the sensors may be placed in locations outside the operating field or room. In a typical scenario, the surgical instrument 150 is operably in communication via a communication medium 160 with various component parts of a “feedback loop” 170. As used herein, the term “feedback loop” includes, but is not limited to, various hardware-software, imaging systems and information systems or other types of information conveying systems that are schematically illustrated in FIG. 1. In addition, in certain embodiments control circuitry 172 may be employed to control or regulate various isolated or inter-connected components in the feedback loop. As used herein the term “control circuitry” includes, but is not limited to, electrical circuitry, regulators, valves, rheostats, silicon chips, resistors, capacitors, transistors etc., that can maintain or regulate over all control or partial control over some component parts or systems in the feedback loop. In one embodiment, control circuitry may process input and output signals from individual or interlinked components. This may include subtraction of input signals or feedback of some output signals into input systems.

Continuing with FIG. 1, one or more sensors 130 can provide information regarding, inter alia, the physical boundary limitations of an operating field. The information may include, but is not limited to, boundary sensing and sensory signals 180. The terms “boundary sensing” and “sensory signals”, as used herein, includes, inter alia, any type of signal that is conveyed into the feedback loop system. In an embodiment, sensory signals include digital or analogue information regarding, for example, images, shapes, landmarks, fiducials, intrinsic characteristics, colors, textures, density, rigidity, moisture content, temperature, pH etc., of anatomical organs. Additionally or alternatively, in some embodiments sensory signals may include boundary information that is provided by intraoperative fluoroscopy, CT scanning, MRI or ultrasound. These would include boundary features that may be defined by tissue density, signal intensity or echogenicity. The anatomical organs may be within or outside the operating field. In alternative embodiments, the boundary sensing and sensory signals may include data, false color or black and white images or information regarding positions of anatomical organs relative to each other (e.g. anatomic positional reference data ) 190. In additional embodiments, the sensors may determine the position or orientation of a surgical instrument 150 relative to an anatomical part(s) or relative to the position of a user(s) of the surgical instrument. Here, a user includes, but is not limited to a surgeon, an operating room personnel, surgical trainer or a robotic user. In some embodiments, the boundary-sensing signals or sensory signals 180 can be delivered to a surgical instrument or a human or robotic user either through a direct hardwired system or through a wireless system. Those skilled in the art will recognize that signals can be conveyed through numerous means. For instance, the means for signal communication may include, but is not limited to radio frequencies, acoustic, ultrasound, electromagnetic, infrared, optical etc. In the communication devices or systems discussed above, hardwired systems or devices may be integrated with or may be a part and parcel of wireless systems or devices.

In accordance with FIG. 1, in an embodiment, image, information, data or sensory signals etc., are transformed into haptic category objects 200. The term “haptic category objects” as used herein, connotes among other things, any virtual object or image that is displayed by means of a computer-assisted device, on a display screen. Haptic category objects may include sound modalities or touch perception modalities. Haptic category objects may include contour mapping data or other types of anatomical geometric or mapping data. In a further embodiment, the feedback system comprises a repository of haptic cues 210 that are in part derived from the haptic category objects 200. The term “haptic cues” encompasses, but not limited to, any information that is available to a human or robotic user 220 that enables the user to produce a user-initiated command 230. Examples of haptic cues may comprise, inter alia, tactile output information, alpha-numeric or numeric signals, audio signals, visual signals or other sensory-based signals that a user may read, feel, touch or hear. A communication medium for communicating haptic cues may include any appropriate signal-carrying path or device such as an optical fiber, a waveguide, a nanotube, a metal wire and/or a nonmetallic wire. According to some embodiments, haptic cues may be made available to a user in real time scale or in nearly real time. By “real time” or “nearly real time” it is understood by persons skilled in the art that these include some temporal delays associated with processing of haptic category objects into haptic cues. The processing may include “instant” processing technology or instant messaging systems.

Returning to FIG. 1, in an embodiment, haptic cues 210 may be stored or fed into a user interface, which may include, but is not limited to, at least one of the following devices: a computer, a key board, a hard drive, a memory, a software or a network 240. The user interface may be tied in to at least one of a hospital information system, a patient monitoring system, a therapeutic plan, a patient history, a treatment plan etc 250. One skilled in the art will appreciate that hospital information system, patient monitoring system, therapeutic plan, patient history, treatment plan etc are, in some embodiments, an integral part of any modern-day patient care systems. These systems include, but are not limited to, diagnostic, data, disease or disorder history, genetic testing databases, sibling genetic history, blood group information, patient family disease history, CT scans, X-ray images, vital sign history etc. Typically, a human or robotic user 220 may access this information via the user interface 240 or through the hospital information system 250 to enact user-initiated commands 230. In an embodiment, user initiated commands may include, but are not limited to, instructions either to activate or inactivate 260 a surgical instrument 150. User-initiated commands may also include instructions to reorient or reposition 260 the surgical instrument. Those skilled in the art will recognize that the activation or inactivation may occur within or outside a physical boundary limitation of an operating field. Furthermore it is conceivable by those skilled in the art that user-initiated commands 230 may include autoinactivation or autoactivation of the surgical instrument 150 may occur abruptly or at regularly phased intervals. In some embodiments, the activation or the inactivation of the surgical instrument 150 can occur through modification of one or more operative characteristics of the surgical instrument 150. Additionally or alternatively, the activation or the inactivation may occur while the surgical instrument 150 is at least partly functioning within an operating field. Also, in some instances, user initiated commands 230 may instruct the surgical instrument 150 to slow down or speed up or change directions. Those skilled in the art will appreciate that numerous methods, protocols, procedures or algorithms are available commercially (or are under research and development) that provide secure patient-care information to authorized users only under conditions of strict privacy. It is therefore understood by those skilled in the art that the herein referred to user-initiated commands 230 in FIG. 1 are maintained and executed under strict privacy and confidentiality.

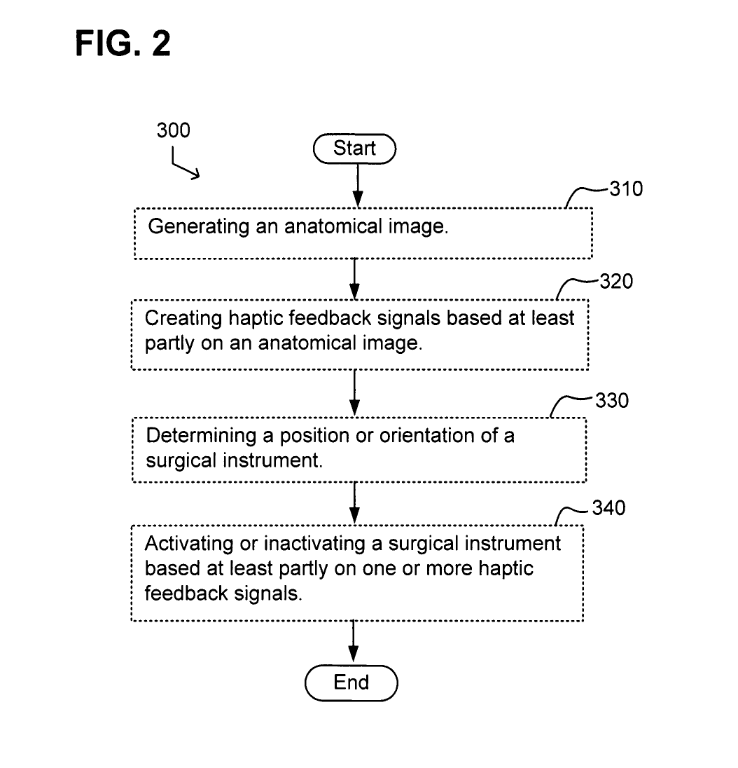

In another aspect of the invention, there is diagrammatically illustrated in FIG. 2, a method of performing haptic surgery. In an embodiment, a typical operational flow of the method 300 includes the following optional steps: (1) generating an anatomical image 310; (2) creating haptic feedback signals based at least partly on the anatomical image 320; (3) determining a position or orientation of a surgical instrument 330; and (4) activating or inactivating the surgical instrument 150 based at least partly on one or more haptic feedback signals 340.

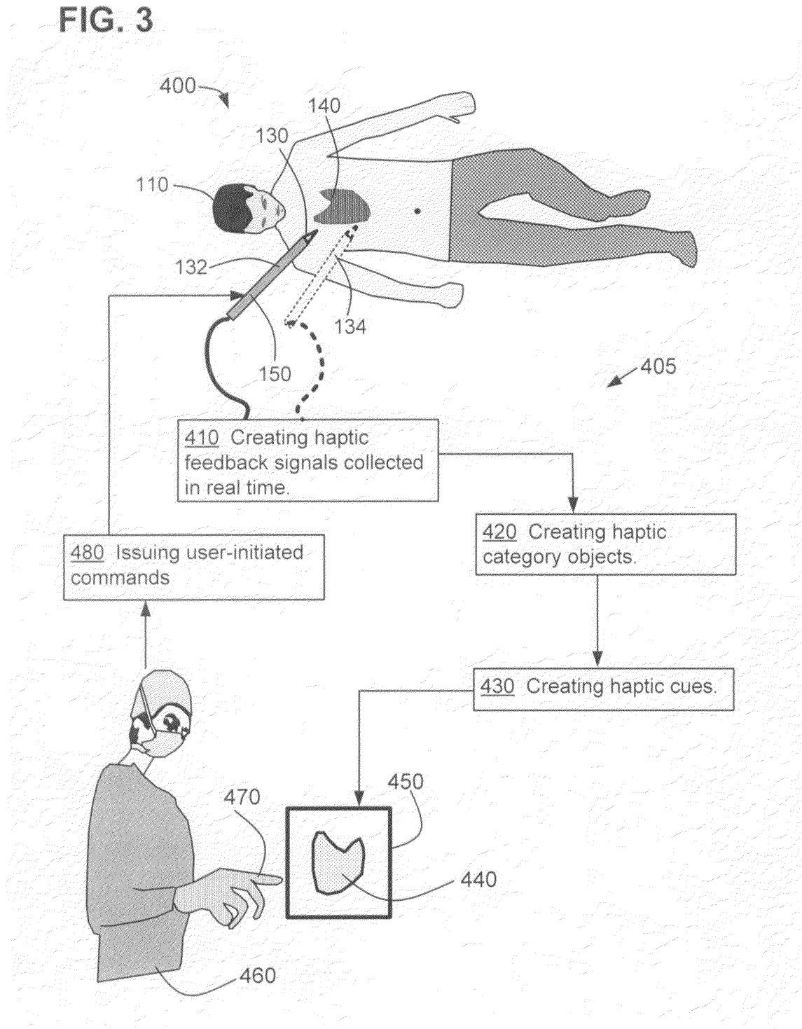

In FIG. 3, there is schematically illustrated a flow chart depicting an embodiment of a method of performing haptic surgery on a patient 110. A typical surgical operational flow 400 includes, inter alia, generating an anatomical image by positioning one or more sensors 130 near a bodily tissue 140. The sensors may be positioned by moving the at least one or more sensors from a first position 132 proximate to a bodily tissue 140 to a second position 134 proximate to the bodily tissue (shown in dotted lines in FIG. 3). Alternatively, the sensors may be used in determining a position or orientation of a surgical instrument 150. In further embodiments, additional operations 405 may be envisaged. These include, inter alia, creating haptic feedback signals in real time 410, partly based on near real time anatomical imaging. In additional or alternative embodiments, haptic feedback signals may be partly based on near-constant acquisition of positional reference data or dynamic boundary-sensing signal states. Those skilled in the art will appreciate that by the term “near-constant acquisition of positional reference data” it is meant, inter alia, that the sensory feedback signals from one or more sensors located in numerous positions near anatomical body parts, are acquired and analyzed at a constant steady-state of signal acquisition, and that the signals are not necessarily processed into images but may be processed as data. Likewise, by the term “dynamic boundary-sensing signal states” it is meant, inter alia, that the boundary-sensing signals may be acquired during, for example, an operation or surgical procedure while one or more anatomical organs or tissues are moving in a state of dynamic flux. Here, the acquired signals may be processed into images or analyzed as data or both.

In alternative embodiments, haptic feedback signals 410 (See FIG. 3) may be partly based on recorded anatomical imaging history of a patient or a class of patients with similar disease histories. Haptic feedback signals may be created from one or more static or dynamic anatomical images from an operating field. In one embodiment, the step of creating haptic feedback signals 410 includes converting one or more images from an operating field into one or more of haptic category objects 420. A further embodiment calls for making haptic category objects 420 available to a user in real time or nearly in real time. Another step in the method 400 includes converting one or more haptic category objects into one or more of haptic cues 430. In another embodiment, the step of creating haptic cues 430 includes binning the one or more haptic cues. Those skilled in the art will recognize that binning involves a search optimization technique that a user may employ. It is based on searching a population of haptic cues according to their intrinsic haptic cue data characteristics. The database of the haptic cue data may be, for example, presorted in order to speed up matching haptic cues captured from haptic category object data from a patient using comparison data from other patients. Referring back to FIG. 3, in an embodiment, the binned or unbinned haptic cues 430 and a haptic category object 440 are made available to a user 460, for example, on a visual display screen 450. In another embodiment, the user 460 may effectuate haptic surgery through the use of a touch-screen method 470 employing tactile cues. Additionally, the user may communicate with a surgical instrument 150 by issuing user-initiated commands 480, which may include, inter alia, activating or inactivating the surgical instrument. Those skilled in the art will recognize that other types of movements such as repositioning the surgical instrument 150, reorientation the surgical instrument, slowing down the surgical instrument 150, increasing the velocity of the surgical instrument may be executed through the user-initiated commands 480.

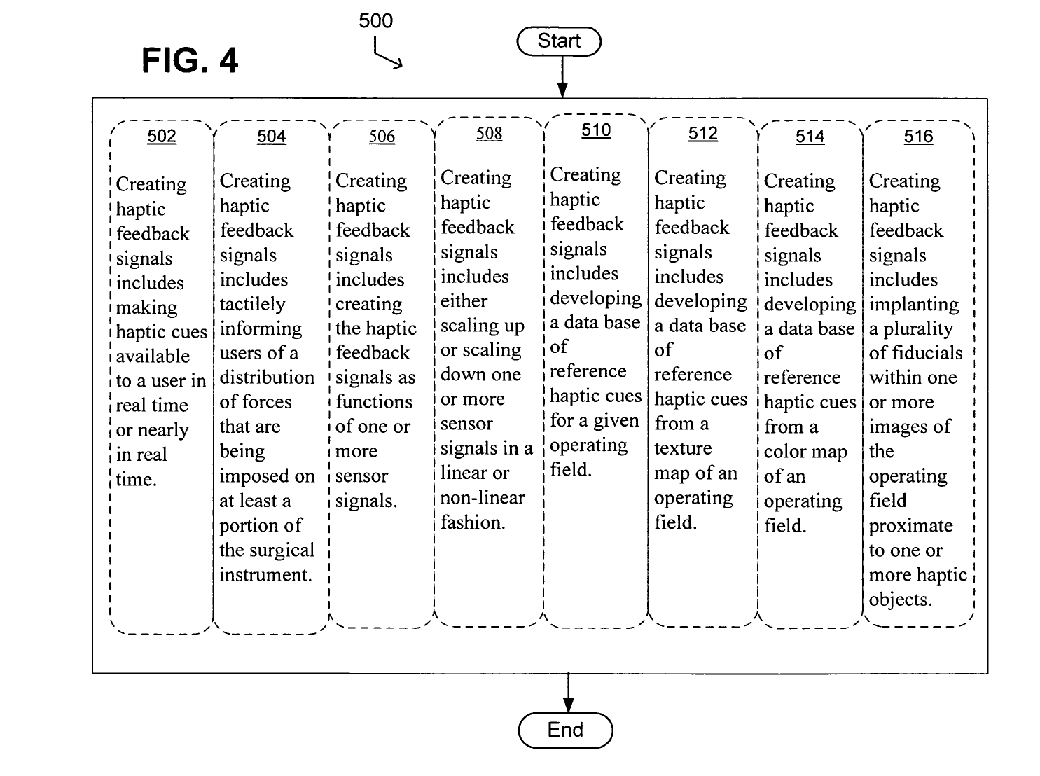

FIG. 4 illustrates a further operational flow 500 for a method of creating feedback signals. In an embodiment, a step 502 of creating haptic feedback signals includes, making haptic cues available to a user in real time or nearly in real time. A creating step 504 may further include tactilely informing users of a distribution of forces that are being imposed on at least a portion of the surgical instrument. Those skilled in the art will recognize that the distribution of forces being imposed on at least a portion of the surgical instrument includes amplitude, frequency, direction, rate of change etc., of the forces. In another embodiment, a step 506 of creating haptic feedback signals includes creating the haptic feedback signals as functions of one or more sensor signals. In another step 508 of the creating haptic feedback signals includes either scaling up or scaling down one or more sensor signals in a linear or non-linear fashion. Optionally, the step 510 of creating haptic feedback signals includes developing a database of reference haptic cues for a given operating field. Additionally or alternatively, the creating haptic feedback signals step 512 may include developing a database of reference haptic cues from a texture map of an operating field. Another embodiment calls for the step 514 of creating haptic feedback signals that includes developing a database of reference haptic cues from a color map of an operating field. The step 516 of creating haptic feedback signals may also include implanting a plurality of fiducials within one or more images of the operating field proximate to one or more haptic category objects.

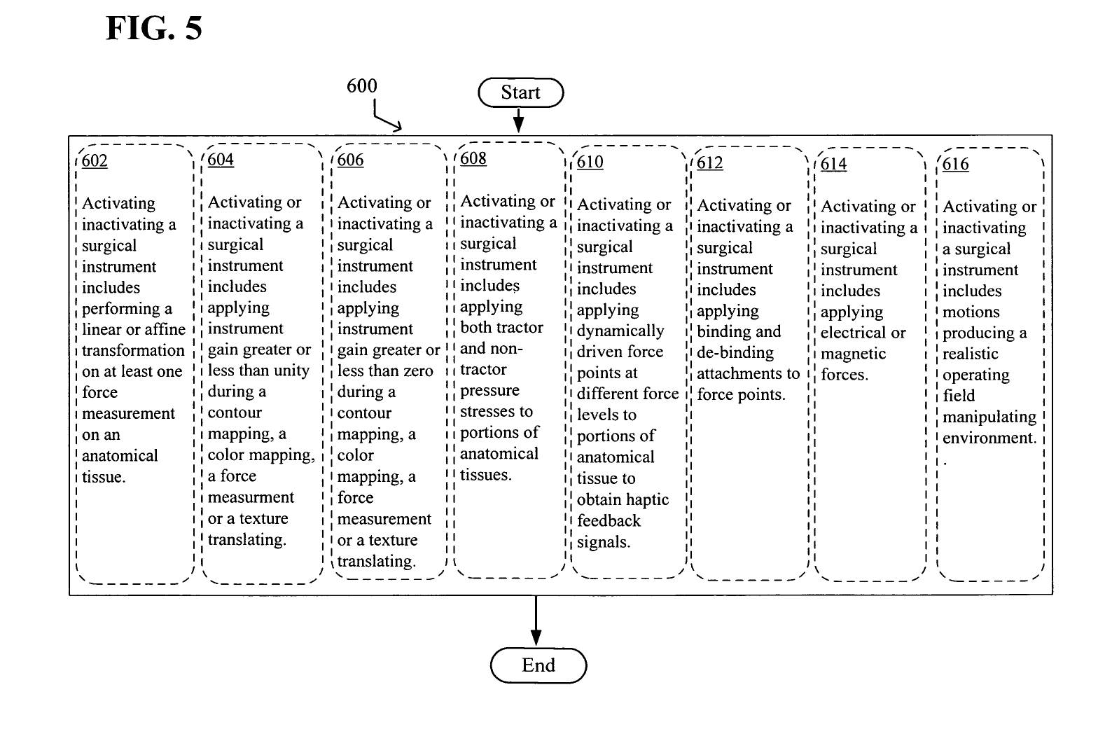

Turning now to FIG. 5, there is illustrated an example of an operational flow 600 for the method of activating or inactivating a surgical instrument. An optional step 602 may include performing a linear or affine transformation on at least one force measurement on an anatomical tissue. Furthermore, the step 604 of activating or inactivating a surgical instrument includes applying instrument gain greater or less than unity during a contour mapping, a color mapping, a force measurement or a texture translating. In an embodiment, the step 606 of activating or inactivating a surgical instrument includes applying instrument gain greater or less than zero during a contour mapping, a color mapping, a force measurement or a texture translating. Another step 608 of activating or inactivating a surgical instrument may include applying both tractor and non-tractor pressure stresses to portions of anatomical tissues. Furthermore, the step 610 of activating or inactivating a surgical instrument includes applying dynamically driven force points at different force levels to portions of anatomical tissue to obtain haptic feedback signals. In a further embodiment, the step 612 of activating or inactivating a surgical instrument includes applying binding and de-binding attachments to force points. Another embodiment, the step 614 of activating or inactivating a surgical instrument includes applying electrical or magnetic forces. In another embodiment, the step 616 of activating or inactivating a surgical instrument includes motions producing a realistic operating field manipulating environment.

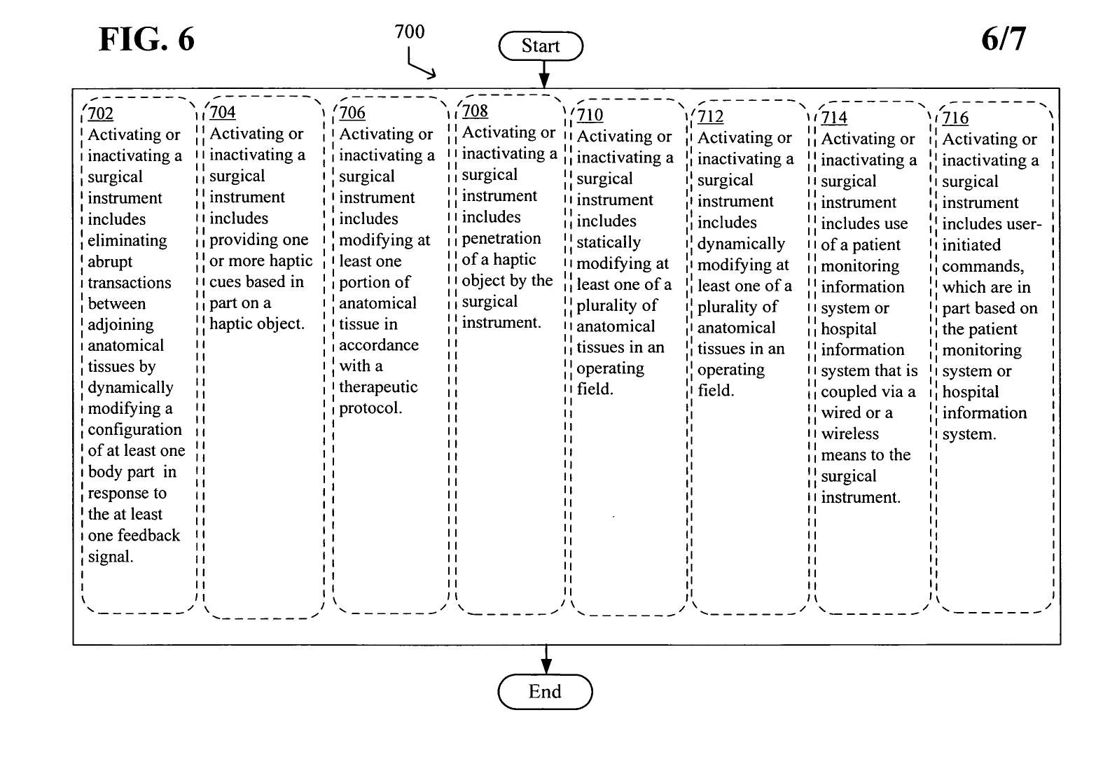

A further aspect of the method of performing haptic surgery is exemplified in FIG. 6 as an illustrative operational flow 700. The method includes, inter alia, a step 702 of activating or inactivating a surgical instrument that includes eliminating abrupt transactions between adjoining or adjacent anatomical tissues by dynamically modifying a configuration of at least one body part in response to the at least one feedback signal. Another embodiment calls for the step 704 of activating or inactivating a surgical instrument that includes providing one or more haptic cues based in part on a haptic object. Another step 706 in the method of activating or inactivating a surgical instrument includes modifying at least one portion of anatomical tissue in accordance with a therapeutic protocol. Additionally or optionally, the step 708 of activating or inactivating a surgical instrument can include penetration of a haptic object by the surgical instrument. Another step 710 of activating or inactivating a surgical instrument includes statically modifying at least one of a plurality of anatomical tissues in an operating field. A further step 712 in the method of activating or inactivating a surgical instrument includes dynamically modifying at least one of a plurality of anatomical tissues in an operating field. There is included a step 714 in the activating or inactivating a surgical instrument, the use of a patient monitoring information system or hospital information system that may be coupled via a wired or a wireless means to the surgical instrument. Optionally, the step 716 of activating or inactivating a surgical instrument includes user-initiated commands, which are in part based on the patient monitoring system or hospital information system. One skilled in the art will appreciate that activating or inactivating a surgical instrument includes turning off the surgical instrument once a user has come close to a given vital organ or anatomical part (e.g., a blood vessel or a nerve). Examples of situations where the surgical instrument may be turned off include (but are not limited to): a cautery losing its cutting current, a sucker losing its suction or a tissue aspirator (also known in the art as CUSA) losing its ultrasonic vibration and suction.

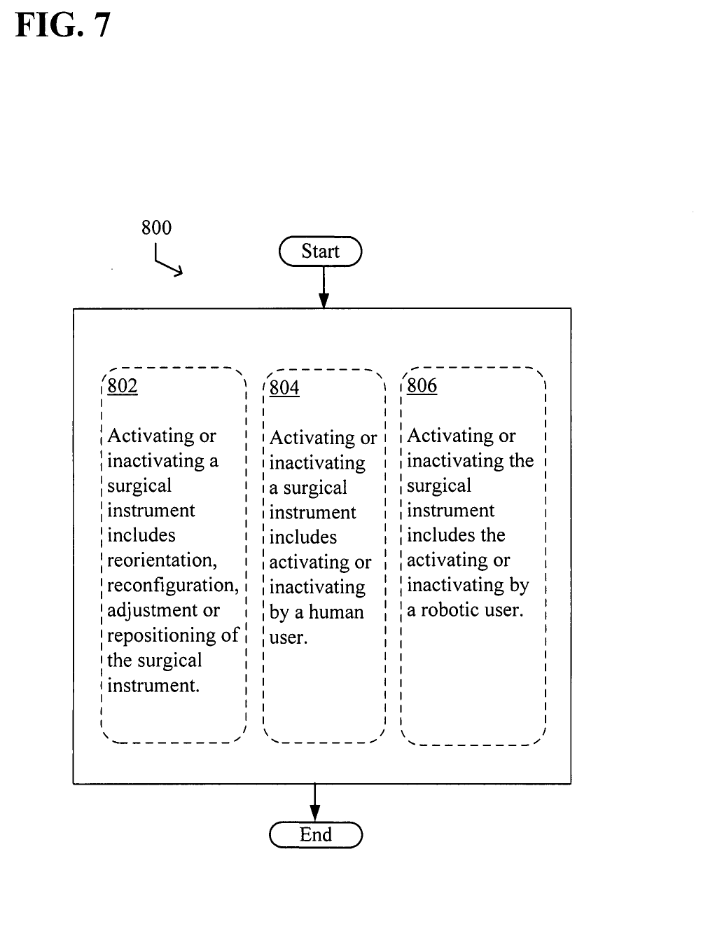

Further embodiments of a method of performing haptic surgery are illustrated in the operational flow 800 depicted in FIG. 7. In one embodiment, the method includes the step 802 of activating or inactivating a surgical instrument, which includes at least one of a reorientation, reconfiguration, adjustment or repositioning of the surgical instrument. Furthermore, the step 804 of activating or inactivating a surgical instrument includes activating or inactivating by a human user. Additionally or alternatively, the step 806 of activating or inactivating the surgical instrument includes the activating or inactivating by a robotic user.

A number of United States patents disclose haptic feedback systems. For example, U.S. Pat. Nos. 5,739,479, 6,494,882, 6,740,058, 7,196,688, 7,204,168 and 7,206,627, which are incorporated herein by reference, disclose devices and methods for performing haptic surgery.

The illustrated devices or methods may be implemented in software, hardware, firmware or combinations thereof. The steps discussed herein need not be performed in the stated order. Several of the steps could be performed concurrently with each other. Furthermore, if desired, one or more of the above described steps may be optional or may be combined without departing from the scope of the present invention.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The foregoing detailed description has set forth various embodiments of the devices or processes via the use of flowcharts, diagrams, figures or examples. Insofar as such flowcharts, diagrams, figures or examples contain one or more functions or operations, it will be understood by those within the art that each function or operation within such flowchart, diagram, figure or example can be implemented, individually or collectively, by a wide range of any combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

The herein described subject matter sometimes illustrates different components contained within, or connected with, different other components. It is to be understood that such depicted figures are merely by way of example, and that in fact many other figures can be implemented which achieve the same functionality. In a conceptual sense, any arrangement of components to achieve the same functionality is effectively “associated” or “coupled” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to, physically mateable or physically interacting components or wirelessly interactable or wirelessly interacting components or logically interacting or logically interactable components.

In a general sense, those skilled in the art will recognize that the various aspects described herein which can be implemented, individually or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory) or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices or processes in the fashion set forth herein, and thereafter use engineering practices to integrate such described devices or processes into image processing systems. That is, at least a portion of the devices or processes described herein can be integrated into an image processing system via a reasonable amount of experimentation. Those having skill in the art will recognize that a typical image processing system generally includes one or more of a system unit housing, a video display device, a memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position or velocity; control motors for moving or distorting lenses to give desired focuses). A typical image processing system may be implemented utilizing any suitable commercially available components, such as those typically found in digital still systems or digital motion systems.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that a limitation is desired.

With respect to the use of substantially any plural or singular terms herein, those having skill in the art can translate from the plural to the singular or from the singular to the plural as is appropriate to the context or application. The various singular or plural permutations are not expressly set forth herein for sake of clarity.

The herein described subject matter sometimes illustrates different components contained within, or connected with, different other components. It is to be understood that such depicted architectures are merely by way of example, and that in fact many other architectures can be implemented which achieve the same functionality. In a conceptual sense, any arrangement of components to achieve the same functionality is effectively “operably coupled” or “coupled” or “in communication with” or “communicates with” or “operatively communicate” such other objects that the desired functionality is achieved. Hence, any two components herein combined to achieve a particular functionality can be seen as associated with each other such that the desired functionality is achieved, irrespective of architectures or intermedial components. Likewise, any two components so associated can also be viewed as being “connected,” or “attached,” to each other to achieve the desired functionality, and any two components capable of being so associated can also be viewed as being “operably couplable,” to each other to achieve the desired functionality.

While particular aspects of the present subject matter described herein have been shown and described, it will be apparent to those skilled in the art that, based upon the embodiments herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.

1. A surgical system, comprising:
a feedback system having one or more sensors operably responsive to physical boundary limitations of an operating field;
wherein the one or more sensors provide information regarding the physical boundary limitations of the operating field; and
a surgical instrument that is configured to respond to the information by either activation or inactivation.

2. The surgical system of claim 1, wherein the activation or the inactivation occurs within the physical boundary limitations of the operating field.

3. The surgical system of claim 1, wherein the activation or the inactivation occurs without the physical boundary limitations of the operating field.

4. The surgical system of claim 1, wherein the activation or the inactivation occurs through modification of one or more operative characteristics of the surgical instrument.

5. The surgical system of claim 1, wherein the information provided by the one or more sensors includes at least one boundary-sensing signal.

6. The surgical system of claim 5, wherein the at least one boundary-sensing signal includes one of a magnetic resonance signal, a computed tomography signal, a computed axial tomography signal, an X-ray imaging signal or an optical imaging signal.

7. The surgical system of claim 5, wherein the at least one boundary-sensing signal is delivered to the surgical instrument.

8. The surgical system of claim 5, wherein the at least one boundary-sensing signal is co-delivered or simultaneously delivered to one or more users.

9. The surgical system of claim 7, wherein the at least one boundary-sensing signal is delivered to the surgical instrument via a direct connection that includes a hardwired system.

10. The surgical system of claim 7, wherein the boundary-sensing signals are delivered to the surgical instrument via a wireless system.

11. The surgical system of claim 1, wherein the activation or the inactivation occurs while the surgical instrument is at least partly functioning within the operating field.

12. The surgical system of claim 1, wherein the one or more sensors is configured to determine at least one orientation or position of the surgical instrument relative to the operating field.

13. The surgical system of claim 1, wherein the one or more sensors is configured to determine at least one orientation or position of the surgical instrument relative to a human or robotic user.

14. The surgical system of claim 1, wherein the activation or the inactivation occurs in operable communication with an instrument positioning system.

15. The surgical system of claim 1, wherein the surgical instrument includes at least one of an endoscope, a dissector, a scalpel, a laser scalpel, a knife, a blade, a needle, a catheter, a scissors, a cutter, a grasper, a surgical tool, a driver, a drill, a saw, a clamper, a pulverizer/crusher, a grinder, a trocar device, a suturer or a stapler, a sucker, a suction device, a cauterizing instrument, a retractor or a probe.

16. A method of performing surgery, comprising:
generating an anatomical image or anatomic positional reference data;
creating haptic feedback signals based at least partly on the anatomical image or the anatomic positional reference data;
determining a position or orientation of a surgical instrument; and
activating or inactivating the surgical instrument based at least partly on the haptic feedback signals.

17. The method of claim 16, wherein the generating an anatomical image or anatomic positional reference data includes positioning one or more sensors in an operating field.

18. The method of claim 17, wherein the determining a position or orientation of a surgical instrument includes positioning at least one sensor in an operating field by moving the at least one or more sensors from a first position proximate to a bodily tissue to a second position proximate to the bodily tissue.

19. The method of claim 16, wherein the creating haptic feedback signals is partly based on near real time anatomical imaging or anatomical information.

20. The method of claim 16, wherein the creating haptic feedback signals is partly based on near-constant acquisition of positional reference data.

21. The method of claim 16, wherein the creating haptic feedback signals is partly based on dynamic boundary-sensing signal states.

22. The method of claim 16, wherein the creating haptic feedback signals is partly based on recorded anatomical imaging history.

23. The method of claim 16, wherein the generating an anatomical image or anatomic positional reference data includes collecting one or more static images or anatomic positional reference data from an operating field.

24. The method of claim 16, wherein the generating an anatomical image includes collecting one or more dynamic images or anatomic positional reference data from an operating field.

25. The method of claim 16, wherein the creating haptic feedback signals includes converting one or more images or anatomic positional reference data from an operating field into one or more of haptic category objects.

26. The method of claim 25, wherein the one or more haptic category objects are provided to a user in real time or nearly in real time.

27. The method of claim 25, wherein the creating haptic feedback signals includes converting one or more haptic category objects into one or more of haptic cues.

28. The method of claim 27, wherein the creating haptic feedback signals includes binning the one or more haptic cues.

29. The method of claim 27, wherein one or more haptic feedback signals are provided to a user in real time or nearly in real time.

30. The method of claim 16, wherein the creating haptic feedback signals includes tactilely informing a user of a distribution of forces being imposed on at least a portion of the surgical instrument.

31. The method of claim 30, wherein the distribution of forces being imposed on at least a portion of the surgical instrument includes amplitude, frequency, direction, rate of change of the forces.

32. The method of claim 16, wherein the creating haptic feedback signals includes the creating the haptic feedback signals as a function of one or more sensor signals.

33. The method of claim 32, wherein the creating haptic feedback signals includes either scaling up or scaling down the one or more sensor signals in a linear or non-linear fashion.

34. The method of claim 32, wherein the creating haptic feedback signals includes either increasing or decreasing signal gain/noise ratio from the one or more sensor signals in a linear or non-linear fashion.

35. The method of claim 16, wherein the creating haptic feedback signals includes developing a database of reference haptic cues for a given operating field.

36. The method of claim 16, wherein the creating haptic feedback signals includes developing a database of reference haptic cues from a texture map of an operating field.

37. The method of claim 16, wherein the creating haptic feedback signals includes developing a database of reference haptic cues from a color map of an operating field.

38. The method of claim 16, wherein the creating haptic feedback signals includes implanting a plurality of fiducials within one or more images or anatomic positional reference data of the operating field proximate to one or more haptic objects.

39. The method of claim 16, wherein the activating or inactivating the surgical instrument includes performing a linear or affine transformation on at least one force measurement on an anatomical tissue.

40. The method of claim 16, wherein the activating or inactivating the surgical instrument includes applying instrument gain greater or less than unity during a contour mapping, a color mapping, a force measurement or a texture translating.

41. The method of claim 16, wherein the activating or inactivating the surgical instrument includes applying offset greater or less than zero during a contour mapping, a color mapping, a force measurement or a texture translating.

42. The method of claim 16, wherein the activating or inactivating the surgical instrument includes applying both tractor and non-tractor pressure stresses to portions of anatomical tissues.

43. The method of claim 16, wherein the activating or inactivating the surgical instrument includes applying dynamically driven force points at different force levels to portions of anatomical tissue to obtain haptic feedback signals.

44. The method of claim 16, wherein the activating or inactivating the surgical instrument includes applying binding and de-binding attachments to force points.

45. The method of claim 16, wherein the activating or inactivating the surgical instrument includes applying electrical or magnetic forces.

46. The method of claim 16, wherein the activating or inactivating the surgical instrument includes motions producing a realistic operating field manipulating environment.

47. The method of claim 16, wherein the activating or inactivating the surgical instrument includes eliminating abrupt transactions between adjoining anatomical tissues by dynamically modifying a configuration of at least one body part in response to the at least one feedback signal.

48. The method of claim 16, wherein the activating or inactivating the surgical instrument includes providing one or more haptic cues based in part on a haptic object.

49. The method of claim 16, wherein the activating or inactivating the surgical instrument includes modifying at least one portion of anatomical tissue in accordance with a therapeutic protocol.

50. The method of claim 16, wherein the activating or inactivating the surgical instrument includes penetration of a haptic object by the surgical instrument.

51. The method of claim 16, wherein the activating or inactivating the surgical instrument includes statically modifying at least one of a plurality of anatomical tissues in an operating field.

52. (Canceled)

53. The method of claim 16, wherein the activating or inactivating the surgical instrument includes use of a patient monitoring information system or hospital information system that is coupled via a wired or a wireless means to the surgical instrument.

54. The method of claim 53, wherein the activating or inactivating the surgical instrument includes user-initiated commands, which are in part based on the patient monitoring system or hospital information system.

55. The method of claim 54, wherein the user-initiated commands are executed or maintained in a secure confidential manner and are accessible to authorized users only.

56. The method of claim 16, wherein the activating or inactivating the surgical instrument includes reorientation, reconfiguration, adjustment or repositioning of the surgical instrument.

57. The method of claim 16, wherein the activating or inactivating the surgical instrument includes activating or inactivating by a human or robotic user.

58. (cancelled)

59. A surgical system, comprising:
a means for generating an anatomical image or anatomic positional reference data;
a means for creating haptic feedback signals based at least partly on the anatomical image or the anatomic positional reference data;
a means for determining a position or orientation of a surgical instrument; and
a means for activating or inactivating the surgical instrument based at least partly on the haptic feedback signals.
